(19) United States
(12) Patent Application Publication
(10) Pub. No.: US 2026/0221275 A1
(43) Pub. Date: Jul. 30, 2026

(54) INVENTORY SYSTEMS AND METHODS FOR REDUCING VARIABILITY IN RETAINED SURGICAL ITEM DETECTION

(71) Applicant: Covidien LP, Mansfield, MA (US)

(72) Inventors: Rayne Y. ROBERTSON, Firestone, CO (US); Richard L. CROFT, Mead, CO (US)

(21) Appl. No.: 19/145,583

(22) PCT Filed: Jan. 12, 2024
(86) PCT No.: PCT/IB2024/050330

§ 371 Date: Jul. 3, 2025

Related U.S. Application Data
(60) Provisional application No. 63/438,806, filed on Jan. 13, 2023.

(51) Int. Cl. G16H 40/40; A61B 17/34; A61B 90/00; A61B 90/98

(52) U.S. Cl. CPC G16H 40/40; A61B 17/3423; A61B 90/98; A61B 2090/0805

(57) ABSTRACT

An inventory system configured for detecting and counting surgical items includes an RFID tag affixed to a surgical item, a signal generator (120) including a display, a surgical access portal, a processor, and a memory. The RFID tag is configured to transmit a return signal when energized. The surgical access portal (300) includes a cannula defining a passageway configured for accessing a surgical site, and an antenna (110) in communication with the signal generator, the antenna configured to receive the return signal transmitted by the RFID tag. The antenna is supported on the cannula and surrounds the passageway. The memory includes instructions stored on the memory, which when executed by the processor cause the system to energize the RFID tag as the surgical item passes through the passage of the cannula, receive the return signal from the antenna, and provide a count based on the return signal.

This application claims priority from U.S. Provisional Patent Application 63/438,806, filed 13 Jan. 2023, the entire content of which is incorporated herein by reference.

### FIELD

The present disclosure relates generally to interrogation and detection systems for radio-frequency (RF) ID tags, and more particularly, detection and inventory systems for potentially retained surgical items within surgical sites.

### BACKGROUND

Inventory systems are used to determine whether objects associated with a surgery are present in a patient's body before completion of the surgery. Such objects may take a variety of forms. For example, the objects may take the form of instruments, for instance, scalpels, scissors, forceps, hemostats, and/or clamps. Also, for example, the objects may take the form of related accessories and/or disposable objects, for instance, surgical sponges, gauzes, and/or pads.

### SUMMARY

Failure to locate an object before closing the patient may require additional surgery, and in some instances, may have unintended medical consequences. Accordingly, there is a need for a technology or system that is capable of providing both presence detection and tagged surgical item/implement identification functionality in the medical setting, as well as inventory controls of the tagged items/implements. Specifically, detecting the presence of, identifying, and maintaining inventory of tagged surgical items and materials that are used during the execution of a medical procedure. Technologies or systems exist that enable these functions both individually as well as in conjunction with each other, but the methods and packaging of the discrete solutions used are not ideal for the application. More specifically, the components attached or affixed to the items being tracked are either too large physically and present nuisances or obstacles in the execution of the procedure, or the detection and identification performance of the solution may degrade rapidly in the presence of variable and uncontrolled dielectric or conductive materials.

Accordingly, there are needs for improvements in presence detection, tagged item identification, and inventory functionality in the medical setting.

This disclosure relates to systems for detection of surgical objects and/or devices used in body cavities during surgery, specifically systems that include an antenna that is part of a surgical access portal inserted directly in a surgical site to detect such surgical objects and/or devices.

In accordance with aspects of the disclosure, an inventory system configured for detecting and counting surgical items includes an RFID tag affixed to a surgical item, a signal generator including a display, a surgical access portal, a processor, and a memory. The RFID tag is configured to transmit a return signal when energized. The surgical access portal includes a cannula defining a passageway configured for accessing a surgical site; and an antenna in communication with the signal generator, the antenna configured to receive the return signal transmitted by the RFID tag. The antenna is supported on the cannula and surrounds the passageway. The memory stores instructions which, when executed by the processor, cause the system to: energize the RFID tag as the surgical item passes through the passage of the cannula; receive the return signal from the antenna; and provide a count based on the return signal.

In an aspect of the present disclosure, the instructions, when executed by the processor, may further cause the system to determine, based on the return signal and the count, whether the surgical item is either inserted into the surgical site or been removed from the surgical site.

In another aspect of the present disclosure, the instructions, when executed by the processor, may further cause the system to provide an indication that the surgical item is either inserted into the surgical site or is removed from the surgical site based on the determination.

In yet another aspect of the present disclosure, the signal generator may include an indicator configured to indicate when the surgical item is removed from the surgical site.

In a further aspect of the present disclosure, the instructions, when executed by the processor, may further cause the system to provide the indication via the indicator of the signal generator.

In yet a further aspect of the present disclosure, the instructions, when executed by the processor, may further cause the system to: determine that no surgical items remain in the surgical site based on the return signal and the count and provide an all-clear indication based on the determination.

In an aspect of the present disclosure, the instructions, when executed by the processor, further cause the system to: determine that one or more surgical items remain in the surgical site based on the return signal and the count and provide an indication that the one or more items remain in the surgical site based on the determination.

In yet another aspect of the present disclosure, the return signal may include an ID name and/or an ID number. When the surgical item enters the surgical site, the instructions, when executed by the processor, may further cause the system to read the ID name and/or the ID number from the return signal and store the ID name and/or the ID number in memory.

In a further aspect of the present disclosure, when the surgical item is removed from the surgical site, the instructions, when executed by the processor, may further cause the system to: read the ID name and/or the ID number from the return signal and compare the ID name and/or the ID number to the stored ID name and/or the ID number in memory.

In yet a further aspect of the present disclosure, the antenna communicates wirelessly with the signal generator.

In accordance with aspects of the disclosure, a computer-implemented method for detecting and counting surgical items includes energizing an RFID tag by an energizing signal and receiving a return signal from an antenna of a surgical access portal. The surgical access portal is configured for accessing a surgical site. The antenna is configured to receive the return signal transmitted by the RFID tag. The antenna is supported on a cannula of the surgical access portal and surrounds a passageway of the surgical access portal. The method further includes providing an indication that the surgical item has entered the antenna and is either inserted in the surgical site or is removed from the surgical site based on the return signal.

In an aspect of the present disclosure, the method may further include determining, based on the return signal and the count, whether the surgical item is either inserted into the surgical site or been removed from the surgical site.

In another aspect of the present disclosure, the method may further include providing an indication that the surgical item is either inserted into the surgical site or is removed from the surgical site, based on the determination.

In yet another aspect of the present disclosure, the method may further include indicating that the surgical item is removed from the surgical site by an indicator of a signal generator in electrical communication with the antenna.

In a further aspect of the present disclosure, the antenna may communicate wirelessly with the signal generator.

In yet a further aspect of the present disclosure, the method may further include determining that no surgical items remain in the surgical site based on the return signal and the count and providing an all-clear indication based on the determination.

In an aspect of the present disclosure, the method may further include determining that one or more surgical items remain in the surgical site based on the return signal and the count and providing an indication that the one or more surgical items remain in the surgical site based on the determination.

In accordance with aspects of the disclosure, the return signal may include an ID name and/or an ID number. When the surgical item enters the surgical site, the method may further include reading the ID name and/or the ID number from the return signal and storing the ID name and/or the ID number in memory.

In yet another aspect of the present disclosure, when the surgical item is removed from the surgical site, the method may further include reading the ID name and/or the ID number from the return signal and comparing the ID name and/or the ID number to the stored ID name and/or ID number in memory.

In accordance with aspects of the disclosure, a non-transitory computer-readable storage medium in which is stored instructions for causing a processor to execute a computer-implemented method for detecting and counting surgical items is presented. The method includes energizing an RFID tag by an energizing signal and receiving a return signal from an antenna of a surgical access portal. The surgical access portal is configured for accessing a surgical site. The antenna is configured to receive the return signal transmitted by the RFID tag. The antenna is supported on a cannula of the surgical access portal and surrounds a passageway of the surgical access portal. The method further includes, based on the return signal, providing an indication that the surgical item has entered the antenna and is either inserted the surgical site or is removed from the surgical site.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements and have been solely selected for ease of recognition in the drawings.

Various aspects of the presently disclosed systems for detection of surgical objects and/or devices are described hereinbelow with reference to the drawings.

FIG. 1 is a schematic diagram showing a surgical environment illustrating a clinician using an inventory system for detecting and counting an object within a patient that is tagged with an RFID tag according to one illustrated aspect;

FIG. 2 is a block diagram of a controller of the system of FIG. 1;

FIG. 3 is a diagram illustrating a surgical access portal of the system of FIG. 1;

FIG. 4 is a diagram illustrating a signal generator of the system of FIG. 1;

FIG. 5 is a flow chart of a computer-controlled method for detecting and counting potentially retained surgical items using the system of FIG. 1;

FIG. 6 is a flow chart of a computer-controlled method for detecting and counting potentially retained surgical items using the system of FIG. 1; and

FIG. 7 is a flow chart of a computer-controlled method for detecting and counting potentially retained surgical items using the system of FIG. 1.

### DETAILED DESCRIPTION

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

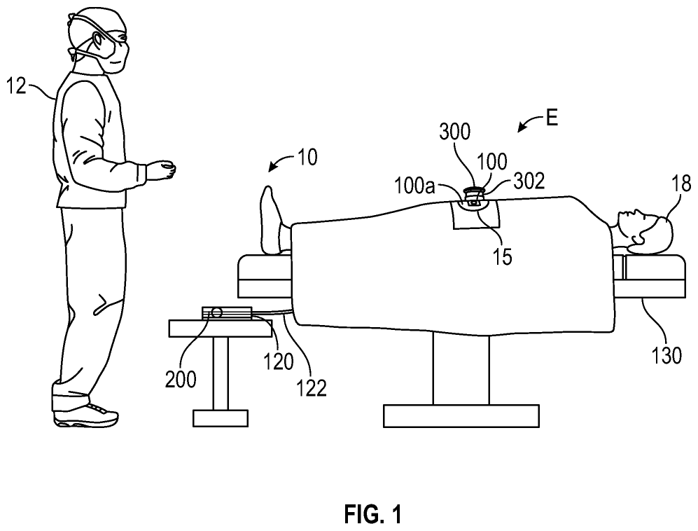

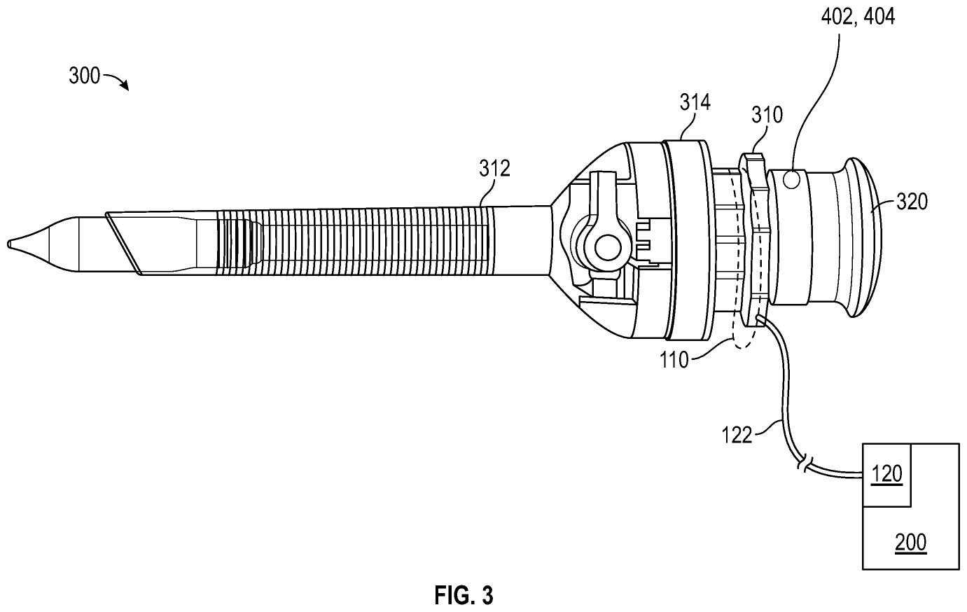

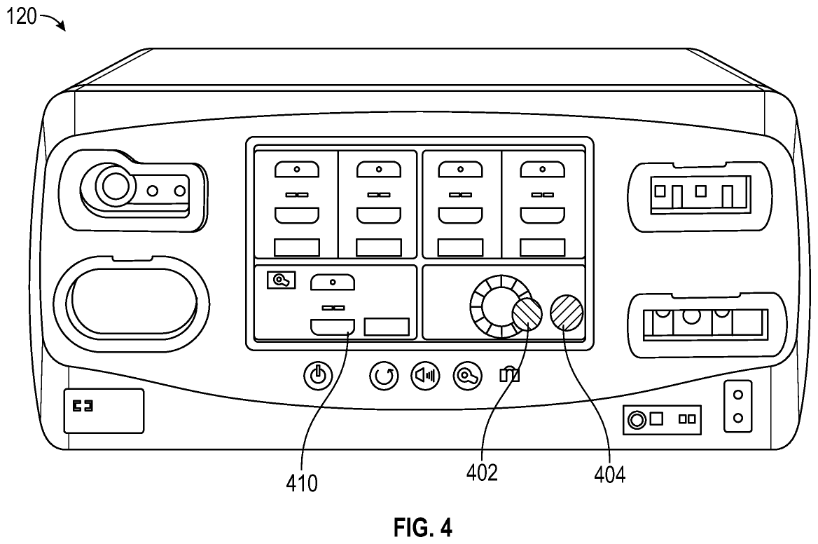

FIG. 1 depicts a surgical environment “E” in which a clinician 12 operates an inventory system 10 for detection and counting of radio-frequency identification (RFID) tags to ascertain the presence or absence of surgical items, implements, or objects 100a in a patient 18. The inventory system 10 includes a surgical access portal 300 (FIG. 3) and a signal generator 120 (FIG. 4). The surgical access portal 300 includes an antenna 110 or cannula assembly coupled to the signal generator 120 by one or more communication paths, for example, coaxial cable 122. Although a cable 122 is shown, the antenna 110 may communicate with the signal generator 120 wirelessly (e.g., WI-FI and/or Bluetooth®).

The object 100a may take a variety of forms, for example, instruments, accessories, and/or disposable objects useful in performing surgical procedures. For instance, the object 100a may take the form of scalpels, scissors, forceps, hemostats, and/or clamps. Also, for example, the objects 100a may take the form of surgical sponges, gauze, and/or padding. The object 100a is tagged, carrying, attached, or otherwise coupled to an RFID tag 100. Aspects of the inventory system 10 disclosed herein are particularly suited to operate with one or more RFID tags 100, which are not accurately tuned to a chosen or selected resonant frequency. Consequently, the RFID tags 100 do not require high manufacturing tolerances or expensive materials and thus may be inexpensive to manufacture.

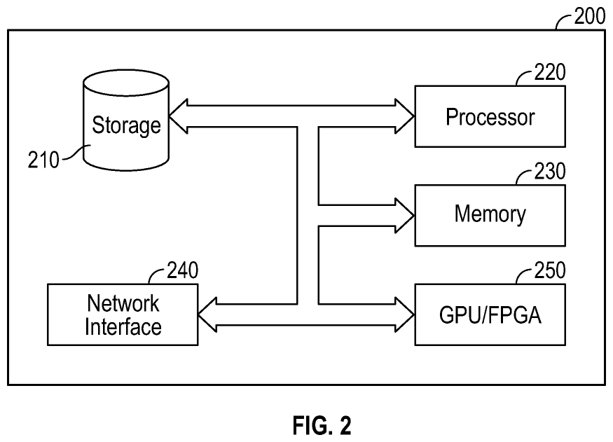

FIG. 2 illustrates that controller 200 includes a processor 220 connected to a computer-readable storage medium or a memory 230. The computer-readable storage medium or memory 230 may be a volatile type of memory, e.g., RAM, or a non-volatile type of memory, e.g., flash media, disk media, etc. In various aspects of the disclosure, the processor 220 may be another type of processor such as, without limitation, a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 includes computer-readable instructions that are executable by the processor 220 to operate the controller 200. In other aspects of the disclosure, the controller 200 may include a network interface 240 to communicate with other computers or to a server. A storage device 210 may be used for storing data.

Referring to FIG. 3, the surgical access portal 300 is shown. The surgical access portal 300 generally includes the antenna 110 disposed within a cannula 310. The surgical access portal 300 may include a removable trocar 320, configured for piercing tissue.

The cannula 310 includes an elongated tubular sleeve 312 defining a passageway and is configured for accessing a surgical site 15, for example during laparoscopic operations. A cannula housing 314 is disposed at a proximal end of the elongated tubular sleeve 312 and defines a passageway that communicates with the passageway of the elongated tubular sleeve 312. During a typical laparoscopic, or minimally invasive procedure, surgical access portals 300, e.g., cannula assemblies, are inserted into the patient's body through the incision in tissue. Surgical instrumentation of varying diameter may then be inserted through the cannula 310 to perform the minimally invasive procedure at the target surgical site 15.

The antenna 110 may be integrated into a side wall of the cannula 310 and/or on an outer surface of the cannula housing 314. The antenna 110 may be a loop antenna configured to determine whether the RFID tag 100 passes through the loop or outside of the loop. The antenna 110 may include a signal source for generating an energizing signal. The antenna 110 may use inductive coupling or capacitive coupling to generate an energizing signal and to read the RFID tag 100.

The surgical access portal 300 may include one or more indicators 402. The indicators may be configured to indicate the object 100a type and/or count. For example, the indicators may include a graphic display, LED lights (e.g., red and/or green lights), and/or an alphanumeric display that indicate a potentially retained item or indicate an all-clear. Although colored lights are used as an example, other forms of indication, such as blinking light patterns, sounds, counts, and/or icons, are contemplated to be within the scope of the disclosure.

Generally, in use, the clinician 12 may introduce or remove an object 100a (e.g., a surgical sponge) from the surgical site 15 via the cannula 310. The antenna 110 may detect the presence (or absence) of the one or more RFID tags 100 and hence that an object 100a has traversed antenna 110 and may have been introduced into the surgical site 15 or removed from the surgical site 15. For a detailed description of an exemplary inventory system, reference may be made to commonly owned U.S. Patent Application Publication No. 2004/0250819, the entire content of which is hereby incorporated by reference herein.

Referring to FIG. 4, the signal generator 120 includes a display 410 and indicators 402, 404. The indicators 402, 404, may be configured to indicate the object 100a type and/or count. For example, the indicators 402, 404 may include red and/or green lights that indicate a potentially retained item or indicate an all-clear. Although colored lights are used as an example, other forms of indication, such as blinking light patterns or icons, are contemplated to be within the scope of the disclosure. The display 410 may indicate additional information about the object 100a, such as an ID name and/or ID number, the specific surgical access portal 300 that is used for insertion and/or retraction of the object 100a from the surgical site 15, or other relevant information about the object 100a. The signal generator 120 may be in wireless communication (e.g., Bluetooth®) with the antenna 110. The signal generator 120 may be operatively coupled to the antenna 110. The signal generator 120 may be configured to generate the energizing signal for the antenna 110.

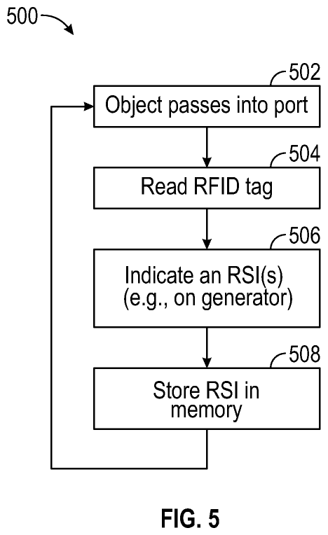

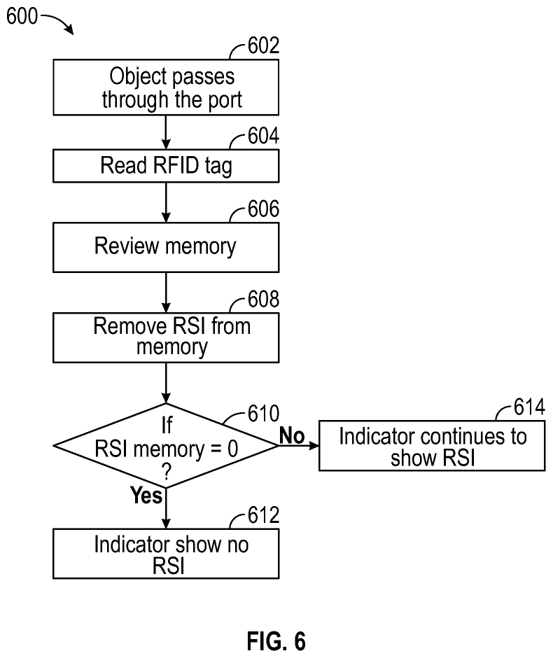

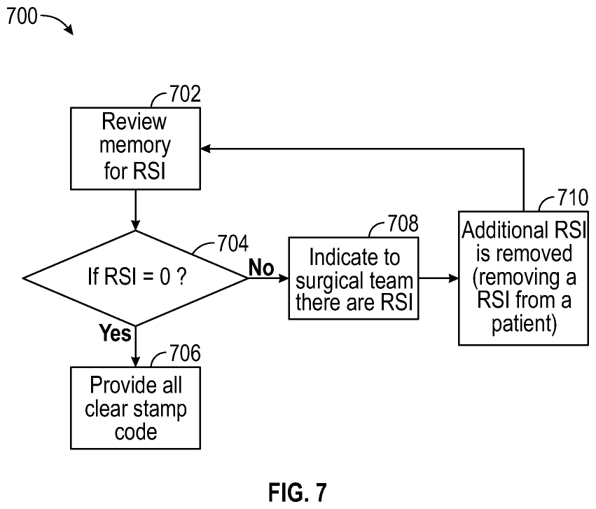

Referring to FIGS. 5-7, there are shown flow charts of exemplary computer-implemented methods 500, 600, 700, for detecting and counting surgical items within surgical sites in accordance with aspects of the present disclosure. Although the steps of FIGS. 5-7 are shown in a particular order, the steps need not all be performed in the specified order, and certain steps can be performed in another order. For simplicity, FIGS. 5-7 will be described below, with the controller 200 performing the operations. However, in various aspects, the operations of FIGS. 5-7 may be performed in part by the controller 200 of FIG. 2 and in part by another device, such as a remote server. These variations are contemplated to be within the scope of the present disclosure.

Initially, at step 502, the object 100a (e.g., a sponge or other surgical item including RFID tag 100) passes into the cannula 310 of the surgical access portal 300 (e.g., port) of a patient 18.

Next, at step 504, the controller 200 causes the inventory system 10 to read the RFID tag 100 affixed to the object 100a using the antenna 110 of the surgical access portal 300. The inventory system 10 may energize the RFID tag 100 as the object 100a passes through the passageway of the cannula 310. The antenna 110 may receive the return signal from the RFID tag 100 and communicate the signal to the controller 200 for further processing. In aspects, the return signal may include information such as the ID name and/or ID number of the object 100a. The ID name and/or ID number of the object 100a may be stored in memory and associated with the RFID tag 100. In aspects, the ID name and/or ID number may be read form a barcode (e.g., a product barcode), by manual entry (e.g., on signal generator 120), and/or accessing from a supply inventory MRP system. F122

Next, at step 506, the controller 200 causes the inventory system 10 to indicate an RSI (retained surgical item), e.g., that object 100a has been inserted in the surgical site 15. For example, the indication may be displayed on the display 410 of the signal generator 120, with an indicator 402, 404 of the signal generator 120, on a user device, and/or on another display. The displayed information may include the ID name and/or ID number (e.g., a lot and/or serial number) of the object 100a. For example, a serial number and model number of an object 100a may be displayed on the signal generator 120 display 410. The inventory system 10 may store a time code in memory for when the object 100a was scanned by the antenna 110.

Next, at step 508, the controller 200 causes the inventory system 10 to store the RSI in memory. For example, the inventory system 10 may increment an RSI counter in memory.

At step 602, the object 100a passes out of the cannula 310. For example, the clinician 12 removes object 100a (e.g., the sponge) from the surgical site 15 via the cannula 310.

Next, at step 604, the controller 200 causes the inventory system 10 to read the RFID tag 100 of the removed object 100a using the antenna 110 of the surgical access portal 300. The inventory system 10 may energize the RFID tag 100 as the object 100a passes through the passageway of the cannula 310 while being removed. The antenna 110 may receive the return signal from the RFID tag 100 of object 100a and communicate the signal to the controller 200 for further processing.

Next, at step 606, the controller 200 causes the inventory system 10 to review the memory registry for an RSI count.

Next, at step 608, the controller 200 causes the inventory system 10 to remove the RSI from memory. The inventory system 10 may decrement the RSI count by one.

In aspects, the antenna 110 may detect RFID tags 100 that are within a detection range of the antenna 110. For example, an object 100a may be inserted down (or removed from) a “non-RFID” surgical access port that is within the detection range of the antenna 110 and be detected by the antenna 110.

The controller 200 may determine if the RSI count in memory is equal to zero or not at step 610. When the RSI count in memory is equal to zero, as in step 612, then the controller 200 causes the inventory system 10 to indicate no RSI. When the RSI count in memory is greater than zero, as in step 614, then the controller 200 causes the inventory system 10 to continue to indicate RSI.

In aspects, the inventory system 10 may store in memory which of a plurality of surgical access portals 300 an object 100a was used for insertion of the object 100a. The inventory system 10 may store in memory which of the plurality of surgical access portals 300 an object 100a was used for extraction of the object 100a. The inventory system 10 may indicate which of the of surgical access portals 300 was used for insertion and/or extraction of the object 100a. For example, during a laparoscopic surgical operation a patient 18 may have more than one surgical portal 300 inserted in different locations of the surgical site 15, for example different locations in the abdomen. A clinician may insert an object 100a into a first surgical access portal 300 of the more than one surgical access portals 300. The antenna 110 of the first surgical access portal 300 may detect the object 100a is being inserted. The controller 200 may cause the inventory system 10 to store in memory that the object 100a was inserted into the first surgical portal 300. The inventory system 10 may then provide an indication, for example, on a display, that the object 100a was inserted in the first surgical portal 300.

At step 702, the controller 200 may cause the inventory system 10 to review the memory for RSI. At step 704, the controller 200 will determine whether the RSI count in memory is zero or not.

Next, at step 706, if the RSI count in memory is zero, then the controller 200 may cause the inventory system 10 to indicate that there are no RSI. For example, the inventory system 10 may display an “all clear” so that clinicians are aware that no RSI remain. Thus, the disclosed technology has the benefit of enabling the clinician to determine of any material is left behind in the patient 18.

Next, at step 708, if the RSI count in memory is greater than zero, then the inventory system 10 may indicate there are still RSI. The clinical staff would then remove the additional RSI (e.g., object 100a) from the patient 18 (step 710).

While several aspects of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. An inventory system configured for detecting and counting surgical items, the inventory system comprising:
an RFID tag affixed to a surgical item, the RFID tag configured to transmit a return signal when energized;
a signal generator including a display;
a surgical access portal including:
a cannula defining a passageway configured for accessing a surgical site; and
an antenna in communication with the signal generator, the antenna configured to receive the return signal transmitted by the RFID tag, the antenna supported on the cannula and surrounding the passageway;
a processor; and
a memory storing instructions which, when executed by the processor, cause the system to:
energize the RFID tag as the surgical item passes through the passage of the cannula;
receive the return signal from the antenna; and
provide a count based on the return signal.

2. The system of claim 1, wherein the instructions, when executed by the processor, further cause the system to:
determine based on the return signal and the count whether the surgical item is either inserted into the surgical site or is removed from the surgical site.

3. The system of claim 2, wherein the instructions, when executed by the processor, further cause the system to:
provide an indication that the surgical item is either inserted into the surgical site or is removed from the surgical site, based on the determination.

4. The system of claim 3, wherein the signal generator includes an indicator configured to indicate when the surgical item is removed from the surgical site.

5. The system of claim 4, wherein the instructions, when executed by the processor, further cause the system to:
provide the indication via the indicator of the signal generator.

6. The system of claim 1, wherein the instructions, when executed by the processor, further cause the system to:
determine that no surgical items remain in the surgical site based on the return signal and the count; and
provide an all-clear indication based on the determination.

7. The system of claim 1, wherein the instructions, when executed by the processor, further cause the system to:
determine that one or more surgical items remain in the surgical site based on the return signal and the count; and
provide an indication that the one or more items remain in the surgical site based on the determination.

8. The system of claim 1, wherein the return signal includes at least one of an ID name or an ID number, and
wherein when the surgical item enters the surgical site, the instructions, when executed by the processor, further cause the system to:
read the at least one of the ID name or the ID number from the return signal; and
store the at least one of the ID name or the ID number in memory.

9. The system of claim 8, wherein when the surgical item is removed from the surgical site, the instructions, when executed by the processor, further cause the system to:
read the at least one of the ID name or the ID number from the return signal; and
compare the at least one of the ID name or the ID number to the stored at least one of the ID name or the ID number in memory.

10. The system of claim 1, wherein the antenna communicates wirelessly with the signal generator.

11. A computer-implemented method for detecting and counting surgical items, comprises:
energizing an RFID tag by an energizing signal;
receiving a return signal from an antenna of a surgical access portal, the surgical access portal configured for accessing a surgical site, wherein the antenna is configured to receive the return signal transmitted by the RFID tag, and wherein, the antenna supported on a cannula of the surgical access portal and surrounding a passageway of the surgical access portal; and
based on the return signal, providing an indication that the surgical item has entered the antenna and is either inserted in the surgical site or is removed from the surgical site.

12. The computer-implemented method of claim 11, further comprising:
determining based on the return signal and the count whether the surgical item is either inserted into the surgical site or is removed from the surgical site.

13. The computer-implemented method of claim 12, further comprising:
providing an indication that the surgical item is either inserted into the surgical site or is removed from the surgical site, based on the determination.

14. The computer-implemented method of claim 13, further comprising:
indicating that the surgical item is removed from the surgical site by an indicator of a signal generator in electrical communication with the antenna.

15. The computer-implemented method of claim 14, wherein the antenna communicates wirelessly with the signal generator.

16. The computer-implemented method of claim 11, further comprising:
determining that no surgical items remain in the surgical site based on the return signal and the count; and
providing an all-clear indication based on the determination.

17. The computer-implemented method of claim 11, further comprising:
determining that one or more surgical items remain in the surgical site based on the return signal and the count; and
providing an indication that the one or more surgical items remain in the surgical site based on the determination.

18. The computer-implemented method of claim 11, wherein the return signal includes at least one of an ID name or an ID number, and
wherein when the surgical item enters the surgical site, the method further comprises:
reading the at least one of the ID name or the ID number from the return signal; and
storing the at least one of the ID name or the ID number in memory.

19. The computer-implemented method of claim 18, wherein when the surgical item is removed from the surgical site the method further comprises:
reading the at least one of the ID name or the ID number from the return signal; and
comparing the at least one of the ID name or the ID number to the stored at least one of the ID name or the ID number in memory.

20. A non-transitory computer-readable storage medium in which is stored instructions for causing a processor to execute a computer-implemented method for detecting and counting surgical items, the method comprising:
energizing an RFID tag by an energizing signal;
receiving a return signal from an antenna of a surgical access portal, the surgical access portal configured for accessing a surgical site, wherein the antenna is configured to receive the return signal transmitted by the RFID tag, and wherein, the antenna supported on a cannula of the surgical access portal and surrounding a passageway of the surgical access portal; and
based on the return signal, providing an indication that the surgical item has entered the antenna and is either inserted in the surgical site or is removed from the surgical site.
